(19) United States
(12) Patent Application Publication
(10) Pub. No.: US 2007/0137651 A1
(43) Pub. Date: Jun. 21, 2007

(54) Visualization esophageal-tracheal airway apparatus and methods

(75) Inventors: Raymond Glassenberg, Wilmette, IL (US); Zebadiah Kimmel, Brookline, MA (US); Gerald J. Sanders, Sonoma, CA (US)

Correspondence Address: LUCE, FORWARD, HAMILTON & SCRIPPS LLP, 11988 EL CAMINO REAL, SUITE 200, SAN DIEGO, CA, 92130, US

(73) Assignee: EZC Medical LLC, San Francisco, CA (US)

(21) Appl. No.: 11/303,343

(22) Filed: Dec. 16, 2005

(51) Int. Cl. A62B 9/06; A61M 16/00

(52) U.S. Cl. 128/207.15; 128/207.14

(57) ABSTRACT

Airway apparatus and method of use are provided, in which the airway device includes a dual lumen airway having imaging apparatus, self-inflating balloons, and other sensors, thereby allowing rapid intubation and ventilation.

### FIELD OF THE INVENTION

The present invention relates to airway apparatus equipped with visualization capabilities and capable of providing ventilation to the lungs when positioned in either the trachea or the esophagus.

### BACKGROUND OF THE INVENTION

In emergency medical management of a patient, it is essential that a patient airway be established in as short of a time as possible. As is per se known in the art, endotracheal intubation is a common form of providing an airway and administering gaseous medication. Through a properly established airway, air or oxygen can be delivered to the patient in an emergency situation.

One problem that is routinely faced when attempting to provide endotracheal intubation is the difficulty in properly positioning the endotracheal tube. Often the endotracheal tube is improperly placed in a patient's esophagus. When this improper positioning occurs, air, oxygen, or other gas is delivered into the stomach. This improper delivery may deprive the lungs of ventilation and lead to brain damage or death to the patient.

A well-known and often-practiced method of intubation involves the use of a laryngoscope to visualize the laryngeal opening, commonly using a curved Macintosh blade or a straight Miller blade. Once the larynx is visualized, an airway device can be introduced into the trachea. As compared to blindly intubating an airway device into a patient, this procedure reduces the likelihood of improperly positioning the airway device into the esophagus. Nevertheless, use of a laryngoscope presents other risks.

Using an laryngoscope to intubate may result in a multitude of undesired results, such as inadvertent damage to the teeth, injuries to the nose, and lacerations to the lips, tongue, and other areas. Accordingly, it would be desirable to provide an airway device that is less dependant on a laryngoscope.

Previous attempts have been made at developing a ventilation device that can be introduced “blindly”, or without a laryngoscope. These attempts have led to the development of airway devices having two lumens. One example is a device sometimes referred to as a “Combitube,” such as described in U.S. Pat. Nos. 4,688,568 and 5,499,625 to Frass, et al., which are hereby incorporated by reference in their entireties. Those devices may be used for “blind intubation” in which they are inserted orally and may be placed in either the trachea or the esophagus.

One disadvantage with this type of design is the inability to ascertain whether the device is in the trachea or the esophagus. One manner in attempting to determine the proper placement is to auscultate the patient while attempting to provide ventilation through either one or both of the lumens. This method may not be effective when significant ambient noise exists, such as in the back of a moving ambulance operating with sirens.

Another method to attempt to verify placement of a dual lumen airway is to use a Toomey syringe to apply suction to each of the lumens. In theory, greater resistance is felt in esophageal placement. In practice, the resistance may vary from patient to patient. As a result, the user may improperly identify the placement of the device and ventilate through the wrong lumen.

When a patient is ventilated through the wrong lumen of a dual lumen airway device, the patient may suffer brain injury or death by asphyxiation. Additionally, even if a user is able to properly determine the position of a dual lumen airway, it is possible that the device's position may change if not properly inserted a sufficient distance and the patient is subsequently moved.

Given the disadvantages of the known art, it is desirable to provide an airway device and method that is capable of positioning without the need to use a laryngoscope.

It is further desirable to provide an airway device and method that allows for ventilation when the device is placed in either the trachea or the esophagus.

It is yet further desirable to provide a device that can allow the operator to determine the placement of the device without the need to auscultate or use a Toomey syringe.

It is still further desirable to provide an airway device that allows the operator to monitor the position of the airway as it is being used.

### SUMMARY OF THE INVENTION

In view of the above-listed disadvantages with the prior art, it is an object of the present invention to provide an airway device that is capable of being introduced without the necessity of a laryngoscope.

It is another object of the present invention to provide an airway device that can be inserted into either the trachea or the esophagus.

It is a further object of the present invention to provide an airway device that can allow the operator to determine the placement of the device without the need to auscultate or use a Toomey syringe.

It is a further object of the present invention to provide an airway device that allows the operator to monitor the position of the airway as it is being used.

These and other advantages can be accomplished by providing an airway device having two lumens and a visualization device for allowing internal visualization of the intubation procedure and monitoring of the placement.

The airway device of the present invention comprises two lumens allowing ventilation either laterally or through the distal end (furthest from the user). The airway device further comprises a visualization device mounted such that it gathers images along a lateral portion of the device. The visualization device preferably is a camera, such as a CMOS or CCD.

Illumination devices may also be incorporated into the airway to assist the visualization device. Examples of illumination devices include light emitting diodes (LEDs) and infrared lights.

Dual lumen airway devices typically include two lumens that terminate in a common distal end. One lumen is open at the distal end, whereas the other lumen vents laterally and has no exit ports immediately near the distal end. Accordingly, in the laterally-venting lumen, there is a significant amount of unused space between the distal end and the distalmost lateral opening. The present invention takes advantage of that unused space to place visualization and/or illumination components. The result is that there may be little to no increase in the overall delivery profile of the airway device.

The visualization device may transmit signals through a wire or using wireless technology. Signals are received by an imaging device, such as a monitor, where the image may be observed by the operator or other individual.

Observation of the imaging device may allow the user to determine whether the airway device is placed in the esophagus or in the trachea as the airway device is inserted into the patient. Furthermore, the display may be observed for changes, such as may occur when the airway device is inadvertently repositioned as might occur when a patient is moved. These changes may indicate that the airway device is no longer properly positioned, thereby allowing the user to reposition the device before the patient suffers consequential harm.

In accordance with one aspect of the present invention, the dual lumen airway device is disposable and discarded after a single-use. The visualization device includes electrical lead wires that terminate in a connector that may be coupled to a reusable unit that processes the signals from the visualization device to generate images. Preferably, the airway device may be coupled to a reusable module that houses components for powering the visualization device, processing the signals generated by the visualization device, and optionally, powering the illumination device. The reusable module also may include a screen for displaying the images generated by the visualization device, or may generate an output suitable for display on a conventional display.

### BRIEF DESCRIPTION OF THE DRAWINGS

The above and other objects and advantages of the present invention will be apparent upon consideration of the following detailed description, taken in conjunction with the accompanying drawings, in which like reference numerals refer to like parts throughout, and in which:

FIG. 1 is a side view of an embodiment of an airway device incorporating features of the invention;

FIG. 2 is a cross-sectional view of the embodiment of an airway device taken along line 2-2 shown in FIG. 1;

FIG. 3 is a side view of an embodiment of an airway device incorporating features of the invention; and

FIGS. 4A-C depict steps in a method of using the embodiment of the present invention depicted in FIG. 3.

### DETAILED DESCRIPTION OF THE INVENTION

The present invention is directed at a dual lumen airway device that comprises a visualization device that can assist in determining the placement of the device and identifying any subsequent repositioning. These features allow the user to position the device and determine whether the device is placed in the patient's trachea or esophagus in less time than known dual lumen airways. Accordingly, the user can properly ventilate the patient's lungs in a lesser amount of time, thereby increasing patient survivability. The ability of the user to continually monitor the airway's position reduces the risk of an inadvertent repositioning remaining unnoticed.

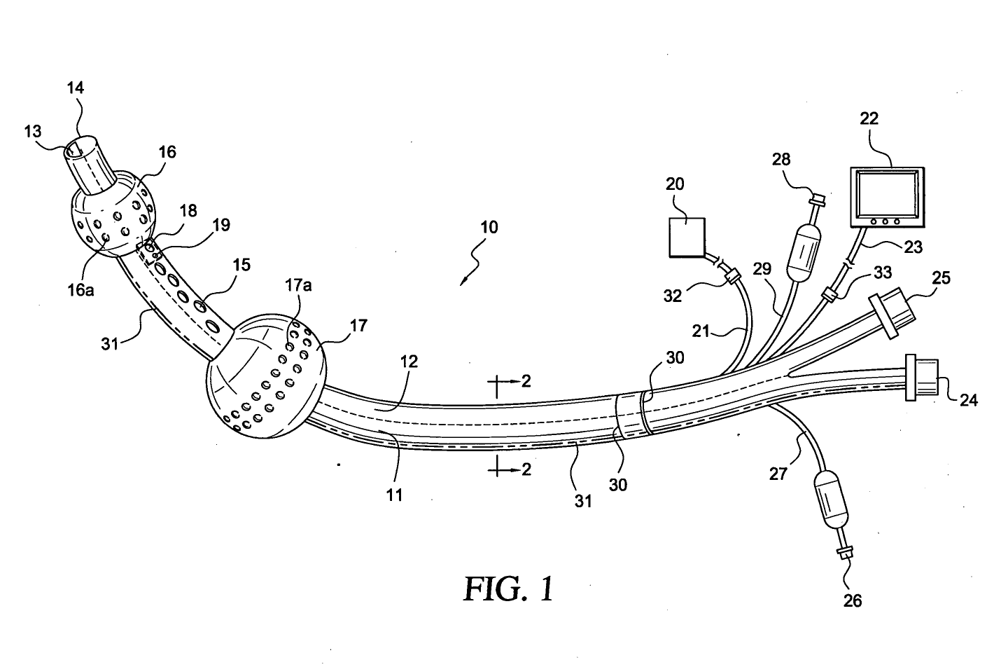

FIG. 1 depicts a preferred embodiment of the present invention. Device 10 has tracheal lumen 11 and esophageal lumen 12. Aperture 13 of tracheal lumen 11 is located at distal end 14 of device 10. Apertures 15 of esophageal lumen 12 are located between distal balloon 16 and proximal balloon 17.

In this embodiment, balloons 16 and 17 comprise texture 16a and 17a. Texture 16a and 17a preferably comprises dimples or indentations, but may also comprise other geometries such as annular channels. Texture 16a and 17a may enhance the interaction between a bodily lumen and balloons 16 and 17. In particular, when balloons 16 and 17 are inflated, the exterior of balloons 16 and 17 will be in contact with the interior of a bodily lumen. Texture 16a and 17a may then be associated with areas of localized suction or increased contact between the interior of the bodily lumen and balloons 16a and 17a.

Device 10 further comprises visualization device 18 located at least partially between distal balloon 16 and proximal balloon 17. In a preferred embodiment, visualization device 18 comprises a CMOS chip, and more preferably comprises a CMOS chip with analog output that can directly interfaced with video hardware using NTSC/PAL format. CMOS chips with analog output that can be directly interface with video hardware using NTSC/PAL format are commercially available, such as models OV7940 and OV7941 available through OmniVision Technologies, Inc., of Sunnyvale, Calif.

Visualization device 18 is preferably configured to reduce the delivery profile of device 10. In particular, visualization device 18 may be configured with a pixel array or other image gathering component remote from the supporting circuitry. By configuring visualization device 18 as described, the circuitry may be positioned in esophageal lumen 12 distal of apertures 15 in space that may otherwise remain unused, as described in greater detail below. The circuitry may be disposed on a conventional circuit board being relatively rigid or may be disposed on a printed circuit board, as is known in the art.

In a preferred embodiment, visualization device 18 provides analog output readable by hardware using NTSC/PAL technology. Hence, the absence of an analog-to-digital converter reduces number of required components incorporated into visualization device 18. Visualization device 18 further may be reduced in size by omitting any infrared filter that would otherwise be commonly associated with a CMOS chip.

In an alternative embodiment, visualization device 18 may comprise a CMOS chip, such as a ⅓ inch CMOS chip or smaller, as is known in the art and is commercially available. The imaging portion of visualization device 18 preferably is embedded or potted in the wall of esophageal lumen 12 and is separated from the outside environment by an optically clear window.

As balloons 16 and 17 are inflated, device 10 typically becomes aligned near the centerline of the trachea or esophagus. As a result, visualization device 18 will be positioned at a distance from the interior wall of the bodily orifice that is geometrically related to the diameter of balloons 16 and 17. As such, visualization device 18 may be selected such that it has a focal length appropriate for the distance that it will be offset from the interior wall of the bodily lumen. Alternatively, visualization device 18 may have a focal length that is adjustable by the user.

Illumination device 19 is located in proximity to visualization device 18, such that illumination device 19 provides visible light, infrared light, or other illumination appropriate for visualization device 18. In the embodiment shown, illumination device 19 comprises one or more LEDs.

In some embodiments, illumination device 19 comprises two or more LEDs that emit light in different wavelengths or at different times. In those embodiments, visualization device 18 may comprise one or more sensors capable of receiving the emitted wavelengths and may be coupled to an analytical device for reconstructing the images.

Power source 20 provides power for visualization device 18 and illumination device 19. Power source 20 as shown comprises an external source of electricity. In other embodiments, power source 20 may comprise an onboard battery. Power source 20 supplies power to, and is in communication with, visualization device 18 and illumination device 19 through conduit 21. Conduit 21 may be an insulated electrical wire or other appropriate medium for transferring energy..

Visualization device 18 is in communication with image display 22 through conduit 23. In other embodiments, visualization device 18 is in communication with image display wirelessly, such as by radio waves, infrared signals, or other known means of wireless communications. Image display 22 preferably converts the signals generated by visualization device 18 into a video image that may be displayed on a viewing screen. Image display 22 for converting the output of a CCD or CMOS chip to a video image are known in the art, and may be of the type commonly used in digital video camcorders. Image display 22 may comprise any suitable video display and may be either integral with, or separate from, power source 20.

Other features of device 10 shown in the embodiment of FIG. 1 include ventilation ports 24 and 25, used to attach an Ambu bag or other ventilation device to tracheal lumen 11 or esophageal lumen 12, respectively. Also, inflation port 26 is in communication with proximal balloon 17 through lumen 27, and inflation port 28 is in communication with distal balloon 16 through lumen 29. Balloons 16 and 17 may be selectively inflated or deflated through inflation ports 26 and 28. For example, inflation ports 16 and 17 are configured to couple with a conventional syringe such that the syringe may be used to force air into the respective balloon. In a preferred embodiment for an adult patient, distal balloon 16 may be inflated with the addition of 15 ml of air or other fluid, whereas proximal balloon 17 may be inflated with 100 ml of air or other fluid. Balloons 16 and 17 can also be deflated by coupling a syringe to the respective inflation port and retracting the plunger, as is known in the art.

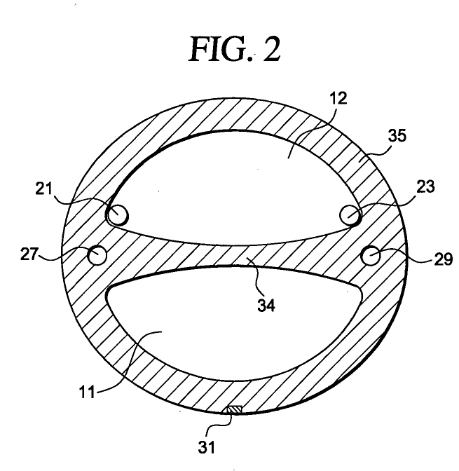

Device 10 also comprises optional markings 30. Markings 30 may comprise circumferential lines, indicia of measurements along an axial direction, or other commonly known system of indicating the proper depth of insertion of device 10. Radio-opaque marker 31 is an optional feature that also may be incorporated into device 10. In this embodiment, radio-opaque marker 31 extends along the axial length of device 10, as seen in FIG. 2.

As is conventional, device 10 is curved and pliable to follow the anatomical structures of a patient.

In accordance with one aspect of the present invention, device 10 is disposable and discarded after a single use. To facilitate this aspect, power connector 32 is disposed along conduit 21 to allow device 10 to be quickly coupled and uncoupled from power source 20 when using an external power supply. Likewise, signal connector 33 is disposed along conduit 23 to allow device 10 to be quickly coupled and uncoupled from image display 22. Image display 22 is a reusable unit that processes the signals from the visualization device 18 to generate images.

Referring now to FIG. 2, the cross section of device 10 taken along line 2-2 as shown in FIG. 1 is depicted. Tracheal lumen 11 and esophageal lumen 12 are separated by divider 34. Conduits 21 and 23 are shown in esophageal lumen 12, but may be located within wall 35 or any other suitable location in other embodiments. Radio-opaque marker 31 and balloon inflation lumens 27 and 29 are located within wall 35 of device 10.

The embodiment shown in FIGS. 1 and 2 takes advantage of space that is underutilized in known dual lumen airways. In this regard, in known designs of dual lumen airways, the esophageal lumen often extends to the distal end of the airway device. Nevertheless, as the ventilation through those esophageal lumens occurs from the ventilation port to the laterally-directed apertures, the space in the esophageal lumen between the apertures and the distal end remains substantially unused. The embodiment depicted in FIGS. 1 and 2 takes advantage of this space by locating a portion of visualization device 18 and/or illumination device 19 in the otherwise vacant space. In embodiments wherein the power supply is an internal battery, the battery may also reside in that space.

When positioning a portion of visualization device 18 in the distal portion of esophageal lumen 12 in device 10, circuitry and other components are preferably located in that area. It is preferable to locate as much of visualization device 18 as possible in the space at the distal portion of esophageal lumen 12 to reduce the volume of the components in the esophageal lumen and allow for a greater airflow.

Conduits 21 and 23 are relatively small compared to the cross sectional area of lumens 11 and 12, and therefore do not prevent adequate ventilation when positioned as shown in FIG. 2.

Device 10 preferably is constructed of a biocompatible clear polymer and is latex-free, although latex or other material may also be used. For adult applications, device 10 preferably has a diameter of 41 French, whereas an alternative embodiment may have a diameter of 37 French for smaller patients.

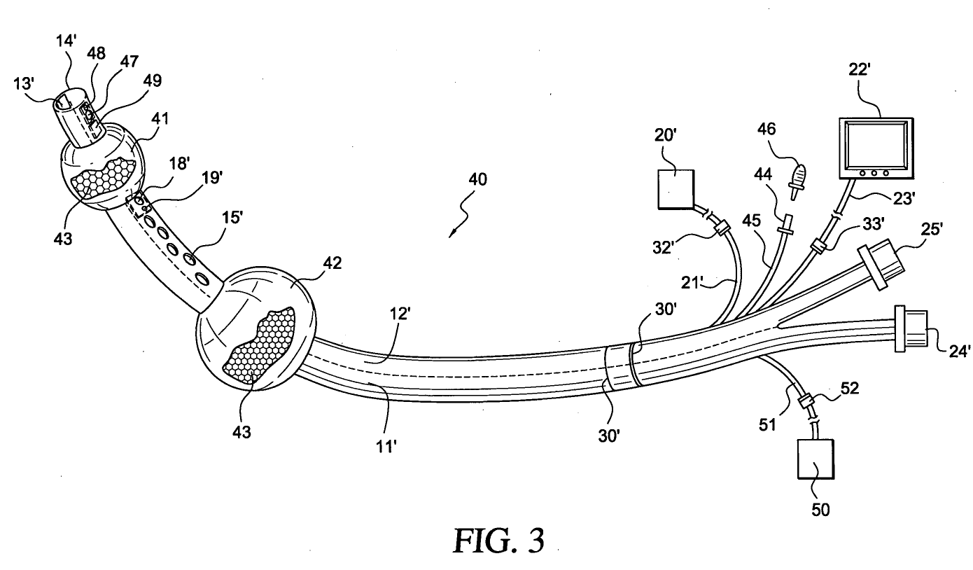

Referring now to FIG. 3, an alternative embodiment of a device in accordance with the present invention is shown. Device 40 is similar to device 10 described above and, accordingly, reference numerals having a prime (′) are similar in description as like numbered components having no prime.

One difference between device 40 and device 10 is the manner in which the apparatus is deployed. In device 10, distal balloon 16 and proximal balloon 17 are inflated by forcing air or other fluid through inflation ports 26 and 28 using a syringe. In contrast, device 40 comprises distal balloon 41 and proximal balloon 42, wherein each balloon surrounds open-cell foam 43 that may be compressed to a small volume when evacuated and that re-expands to conform to and seal the interior of a patient's trachea or esophagus when deployed. One preferred material for open-cell foam 43 is an open-cell polyurethane foam.

Balloons 41 and 42 are connected to port 44 through lumen 45. Port 44 may be obstructed with removable plug 46. When plug 46 is removed, the interior of balloons 41 and 42 are in communication with the environment. Thus, balloons 41 and 42 may be inflated from a compressed configuration by the removal of plug 46, which allows air to reach the interior of balloons 41 and 42, thereby allowing foam 43 to expand.

To deflate previously inflated balloons 41 and 42, a syringe or other suction source may be attached to port 44 to draw air or other fluid from the interior of balloons 41 and 42 and collapse those structures. This deflation may be performed prior to removal of device 40 from a patient.

Device 40 further comprises visualization device 47. Visualization device 47 is preferably disposed within esophageal lumen 12′ near distal end 14′ and distal to apertures 15′. Visualization device 47 preferably is configured to gather images from distal of device 40. Hence, this feature may assist a clinician in determining the placement of the airway as the physician may be able to visualize anatomical landmarks or features, such as rings. Additionally, the clinician may detect repositioning of device 40 by observing a change in anatomical features or landmarks as shown on display 22′.

Visualization device 47 may be used in combination with visualization device 18′ to provide different perspectives of a patient. In other embodiments, visualization device 47 and visualization device 18′ may be positioned in proximity to allow for stereoscopic vision. Visualization device 47 may communicate with display 22′ via conduit 23′, or alternatively may communicate via a second conduit or communicate with a second display.

Device 40 also comprises illumination device 48, which may be similar to illumination device 19′, and may be described in a like fashion.

Additionally, device 40 also may comprise one or more sensors 49. Sensor(s) 49 may be disposed at any convenient location and may comprise carbon dioxide sensors, microphones, nanotube field effect transistors (NTFETs), or other known sensors, and may communicate with output device 50 via conduit 51. Output device 50 may be any appropriate apparatus for communicating information obtained by sensor 49, such as a speaker, digital display, or other known apparatus. Sensor 49 may be coupled and uncoupled to output device 50 via connector 52. In other embodiments, output device 50 may be integral with device 40.

Power source 20′ may be in communication with illumination device 48, visualization device 47, and sensor 49 via conduit 21′. Alternatively, two or more power sources may be used to provide power to the components.

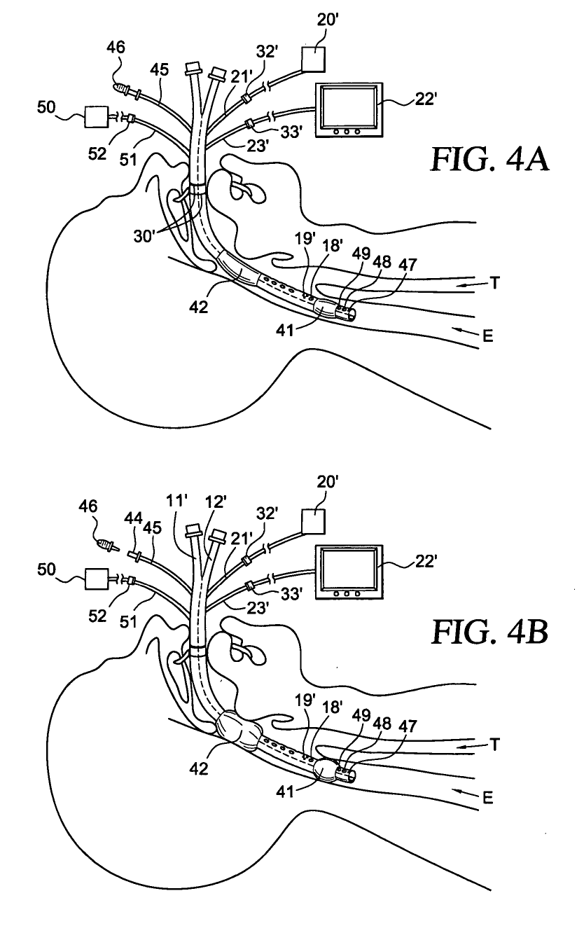

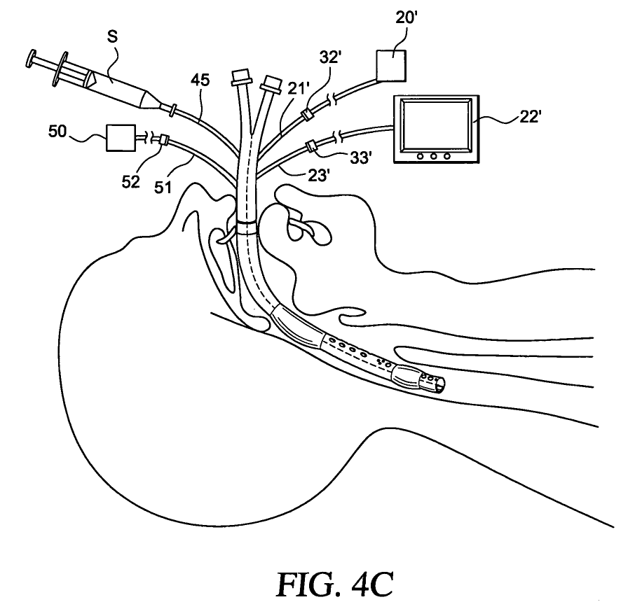

Next, a preferred method of use will be described further illustrating the benefits of the present invention. FIGS. 4 depict several steps in a preferred method of using device 40 described above and depicted in FIG. 3.

Device 40 is preferably stored for use in a sterile container that allows rapid access to device 40. Moreover, balloons 41 and 42 are preferably stored in a collapsed configuration, such that foam 43 is compressed and device 40 has a relatively small delivery profile. Plug 46 is attached to connector 44 at proximal end of conduit 45 to prevent air from reaching the interior of balloons 41 and 42.

To prepare device 40 for use, device 40 is removed from the storage container and examined to ensure that balloons 41 and 42 have not inflated, which may indicate that plug 46 may have become dislodged. Device 40 is connected to display 22′ via connector 33′ on conduit 23′. Device 40 is connected to power supply 20′ via connector 32′ on conduit 21′. Device 40 optionally also may be connected to output device 50 via connector 52 on conduit 51.

The clinician or other individual may observe the output of visualization device 18′ and visualization device 47 on display 22′. Device 40 then may be inserted orally into a patient as the clinician observes display 22′. Device 40 may be distally advanced an appropriate distance, as may be indicated by markings 30′. The clinician may determine whether device 40 is in the patient's trachea T or esophagus E by observing anatomical features and landmarks on display 22′.

In this example, device 40 was placed into the patient's esophagus E, as depicted in FIG. 4A. At this point, the clinician may be aware of the location of device 40 by the output from visualization device 47, which does not show rings, as may be seen with placement in the trachea. Additionally, clinician may be aware of the location of device 40 based on the output of visualization device 18′, which shows the entrance to the larynx. If optional sensor 49 is used, that component may transmit additional information that may be used to determine the position of device 40.

In the event that device 40 was placed in the patient's trachea T, the clinician would have received information to indicate that placement. For example, visualization device 47 may transmit images showing rings consistent with those in the trachea T. Likewise, visualization device 18′ may transmit images that are not taken from the exterior of the entrance to the larynx. Sensor 49 may also transmit different information, such as an increased carbon dioxide reading, increased breath sounds, or other data.

Once device 40 is advanced a sufficient degree, the clinician may inflate balloons 41 and 42 by removing plug 46. After plug 46 is removed, air can travel from the environment, through conduit 45, and into the interior of balloons 41 and 42. As air reaches the interior of balloons 41 and 42, foam 43 expands, thereby inflating balloons 41 and 42 and sealing the bodily lumens in which device 40 is located. This configuration is depicted in FIG. 4B.

After device 40 is deployed by inflating balloons 41 and 42, the clinician may ascertain the position by observing display 22′ and/or output device 50.

If device 40 is positioned in the patient's esophagus E, as shown in FIG. 4B, the clinician may then ventilate the patient via esophageal lumen 12′. This ventilation may be accomplished by attaching an Ambu-bag or other source of air or oxygen to ventilation port 25′. It should be understood that if device 40 was placed in the patient's trachea T, ventilation would occur through tracheal lumen 11′. Advantageously, in either scenario, the clinician need not auscultate the patient or use a Toomey syringe to determine the position of device 40, thereby saving time and allowing oxygen to be delivered to the patient in less time than when using conventional dual lumen airway devices.

Following ventilation of the patient, and any other desired procedures, device 40 may be removed from the patient. Prior to removal, balloons 41 and 42 are preferably deflated. Port 44 preferably is adapted to be coupled to syringe S, which is a conventional syringe. Syringe S is then coupled to port 44 and the plunger is retracted to create suction and withdraw air from balloons 41 and 42 and through conduit 45. FIG. 4C depicts device 40 at a point where syringe S has been attached to port 44 and retracted to deflate balloons 41 and 42. After balloons 41 and 42 are deflated, device 40 may be withdrawn proximally from the patient, thereby completing the ventilation procedure.

It is believed that the operation and construction of the present invention will be apparent from the foregoing description and, while the invention shown and described herein has been characterized as particular embodiments, changes and modifications may be made therein without departing from the spirit and scope of the invention as defined in the following claims.

1. An airway device comprising:
an elongated tube having a proximal end, a distal end, a first lumen extending between the proximal and distal ends, and a second lumen extending at least partially between the proximal end and distal end, the elongated tube defining, near the distal end, at least one aperture connected to the second lumen;
a first balloon circumferentially disposed on the tube distal of the at least one aperture;
a second balloon circumferentially disposed on the tube proximal of the of the at least one aperture;
a visualization device disposed within the tube; and
an illumination device disposed within the tube.

2. The device of claim 1 wherein the visualization device is disposed at least partially within the second lumen.

3. The device of claim 2 wherein the illumination device is disposed at least partially within the second lumen.

4. The device of claim 3 further comprising a self-expanding substance disposed within at least one of the first balloon and second balloon.

5. The device of claim 4 further comprising an inflation lumen in communication with the first balloon and the second balloon.

6. The device of claim 3 further comprising a sensor disposed within the tube.

7. The device of claim 3 wherein the illumination device comprises a first LED that is configured to emit a first wavelength of light and a second LED that is configured to emit a second wavelength of light.

8. The device of claim 3 further comprising a second visualization device.

9. The device of claim 8 wherein the first visualization device is configured to view in a substantially different direction as the second visualization device.

10. An airway device comprising:
an elongated tube having a proximal end, a distal end, a first lumen extending between the proximal and distal ends, and a second lumen extending at least partially between the-proximal end and distal end;
a first balloon circumferentially disposed on the tube near the distal end;
a second balloon circumferentially disposed on the tube proximal of the first balloon, the elongated tube defining one or more apertures disposed between the first balloon and the second balloon and connecting to the second lumen; and
a visualization device disposed at least partially within the second lumen distal to the apertures.

11. The device of claim 10 further comprising an illumination device disposed within the tube.

12. The device of claim 11 wherein the illumination device is disposed at least partially within the second lumen.

13. The device of claim 12 further comprising a self-expanding substance disposed within the first balloon and second balloon.

14. The device of claim 13 further comprising an inflation lumen in communication with the first balloon and the second balloon.

15. The device of claim 14 wherein at least one balloon further comprises a textured exterior surface.

16. The device of claim 12 further comprising a sensor disposed within the tube.

17. The device of claim 12 wherein the illumination device comprises two or more LEDs that are-configured to emit different wavelengths of light.

18. The device of claim 12 further comprising a second visualization device.

19. A method of ventilating a patient comprising:
providing an airway device comprising a first lumen, a second lumen, a visualization device, an illumination device, a first balloon, and a second balloon;
inserting the airway device orally into a patient;
expanding the first balloon;
expanding the second balloon;
receiving data communicated from the visualization device;
determining the position of the airway device based on the data communicated from the visualization device; and
delivering oxygen to the patient through one of the lumens.

20. The method of claim 19 wherein expanding the first balloon and expanding the second balloon comprises allowing air to reach a self-expanding material in the interior of the first balloon and the second balloon.
